(19) United States
(12) Patent Application Publication
(10) Pub. No.: US 2019/0239535 A1
(43) Pub. Date: Aug. 8, 2019

(54) BLACK-EYED PEA PROTEIN ISOLATES, PRODUCTS, AND METHODS

(71) Applicant: Campbell Soup Company, Camden, NJ (US)

(72) Inventor: Keswara Rao Vadlamani, Marlton, NJ (US)

(21) Appl. No.: 16/268,083

(22) Filed: Feb. 5, 2019

Related U.S. Application Data
(60) Provisional application No. 62/626,905, filed on Feb. 6, 2018.

(51) Int. Cl. A23J 1/14; A23L 23/00; A21D 2/26; A23C 9/20; A23C 11/10; C07K 1/36; C07K 1/14; C07K 1/30

(52) U.S. Cl. CPC A23J 1/14; A23L 23/00; A21D 2/266; A23C 9/20; A23V 2002/00; C07K 1/36; C07K 1/145; C07K 1/30; A23C 11/103

(57) ABSTRACT

Embodiments herein include methods for producing black-eyed pea protein isolates and food and beverage products including the same. In an embodiment, a method can include tempering black-eyed pea seeds to 35-45% moisture content and holding 30-120 min, cracking the tempered seeds to loosen the hull and the meat, and separating these two fractions. The method can further include wet-grinding the meat fraction to a finer mash and lowering the pH of the black-eyed pea mash to about 1 to 3 in an aqueous solvent. The method can further include separating an acidic wet cake out from the acidic protein liquid fraction and raising the pH of the acidic wet cake to about 9 to 11 in an aqueous solvent. The method can further include separating alkaline wet cake out from the alkaline protein liquid fraction and combining the acid and alkali protein liquid fractions. Other embodiments are also included herein.

This application claims the benefit of U.S. Provisional Application No. 62/626,905, filed Feb. 6, 2018, the content of which is herein incorporated by reference in its entirety.

### FIELD

Embodiments herein relate to black-eyed pea protein isolates, methods for producing black-eyed pea protein isolates, and food and beverage products including the same.

### BACKGROUND

Increased demand for plant proteins is a key and growing mega trend in the United States. Approximately 40% of new food or beverage products launched in 2015 contain a protein claim. Consumer interest in plant-based diets is expanding. Approximately, 55 million people in the US are considered vegetarian or flexitarian and looking for more plant-based options.

Unfortunately, current plant proteins such as soy, pea and rice have several limitations: they generally do not taste good, soy is considered allergenic, and rice proteins are non-functional (not soluble).

### SUMMARY

Embodiments herein include methods for preparation of black-eyed pea raw material, methods for producing black-eyed pea protein isolates, and food products and beverages including the same.

In an embodiment, a method preparation of black-eyed pea seeds for protein extraction is included herein. The method can include tempering black-eyed pea seeds to 35-60% moisture content and holding for 30-120 minutes, cracking the tempered seeds to loosen the hull and the meat, and separating these into two fractions. The method can further include wet-grinding the meat fraction to a fine mash.

In an embodiment, a method of producing a black-eyed pea protein isolate is included herein. The method can include lowering the pH of the black-eyed pea mash to a pH of about 1.9-2.1 (or about 2) in an aqueous solvent. The method can further include separating an acidic wet cake out from the acid protein liquid portion and retaining the acid protein fraction for further processing.

In an embodiment, the method can include raising the pH of the acidic wet cake to a pH of about 9.8 to 10.2 (or about 10) in an aqueous solvent. The method can further include separating an alkaline wet cake out from the alkaline protein liquid portion and retaining the alkaline protein fraction for further processing. The method can further include combining both acid and alkali protein liquid fractions to provide total protein. The method can further include isoelectric precipitation, concentration, pasteurization, and spray drying.

In an embodiment, a black-eyed pea protein isolate is included herein. The protein isolate can include at least about 70% by weight protein. The protein isolate has a PDCAAS of at least about 0.60.

In an embodiment, a high acid beverage is included. The beverage can include at least about 1 gram of protein per fluid ounce. In an embodiment, the beverage can be at a pH of about 3.5 to about 4.0. In an embodiment, at least 50% of the protein in the beverage is from black-eyed pea protein, the black-eyed pea protein having a PDCAAS of at least about 0.60.

In an embodiment, a low acid beverage is included. The beverage can include at least about 1 gram of protein per fluid ounce. In an embodiment, the beverage can be at a pH of about 6.5 to about 7.0. In an embodiment, at least 50% of the protein in the beverage is from black-eyed pea protein, the black-eyed pea protein having a PDCAAS of at least about 0.60.

In an embodiment, a food product is included. The food product can include at least about 5 grams of protein per serving. In some embodiments, at least 50% of the protein in the food product is from black-eyed pea protein, the black-eyed pea protein having a PDCAAS of at least about 0.60.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood relating to the following drawings, in which:

FIG. 1 is a flow chart showing operations of a method for producing black-eyed pea protein in accordance with various embodiments herein.

FIG. 2 is a flow chart showing operations of a method for producing black-eyed pea protein in accordance with various embodiments here in.

FIG. 3 is a flow chart showing operations in accordance with some embodiments here in.

FIG. 4 is schematic view of a beverage in accordance with various embodiments herein.

FIG. 5 is a schematic view of a food product in accordance with various embodiments herein.

FIG. 6 is a schematic view of a food product in accordance with various embodiments herein.

While embodiments are susceptible to various modifications and alternative forms, specifics thereof have been shown by way of example and drawings, and will be described in detail. It should be understood, however, that the scope herein is not limited to the embodiments described. On the contrary, the intention is to cover modifications, equivalents, and alternatives falling within the spirit and scope herein.

### DETAILED DESCRIPTION

The embodiments described herein are not intended to be exhaustive or to limit the invention to the precise forms disclosed in the following detailed description. Rather, the embodiments are chosen and described so that others skilled in the art can appreciate and understand the principles and practices.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Current plant proteins such as soy, pea and rice have several limitations: they generally do not taste good, soy is considered allergenic, and rice proteins are non-functional (not soluble). As such, there is a need for a cost effective clean-tasting, non-allergenic, and functional plant protein that works well over a wide pH range.

Among the alternate plant protein sources, black-eyed peas offer an attractive raw material with several advantages. They are non-allergenic, gluten free, and sustainable. Black-eyed peas contain 20-25 wt. % protein and low in fat (1 wt. %), and low in saponins. The low-fat content is an added advantage in terms of processing, requiring no extraction with harmful solvents (hexane).

However, the unique shape of black-eyed pea seeds and hull tightly held together by black-eyes pose significant technical challenges in preparation of this raw material for protein extraction. The color from the black-eyes gets extracted during protein isolation process, imparting undesirable darker color, taste, decreasing the overall protein quality and acceptability. To produce high quality black-eyed pea protein, it is imperative to have a starting raw material with little or no black-eye specs. Traditional dry de-hulling techniques such as impact dehullers, roller mills, abrasive peelers etc. used for grains and legumes are not very effective in removing hulls and black-eyes. Moreover, traditional dehulling techniques used for pulses result in 25-30% raw material loss during processing, while still leaving significant number of black-eye specs.

Also, current traditional protein extraction and isolation methods practiced both commercially and in published literature of various plant proteins, teach alkaline pH 8-10 protein solubilization and isoelectric precipitation process to recover protein. This method assumes that plant proteins follow typical solubility curves and majority of seed proteins can be solubilized and recovered by this process. The percent of total seed protein recovery reported in the literature ranges from 35-60% by weight. Typically, about 40-50% of valuable protein is not recovered and is going into waste stream. Thus, there is a real unmet need in the industry to have a viable commercial process that produces a cost effective, high quality black-eyed pea protein.

Embodiments herein include methods for the preparation of black-eyed pea protein which provide a high yield of high-quality protein along with little to no black-eye specs. The total content of protein in black-eyed peas includes two different fractions, wherein the protein in the two different fractions exhibits different physical properties. For examples, the fractions are different in terms of their solubility at different pH values. As such, the fractions obtained from black-eyed peas can include an “acid protein fraction” and a “alkaline protein fraction”. The acid soluble fraction is more soluble at a low pH (for example, a pH of 2). The alkaline soluble fraction is more soluble at alkaline pH (for example, a pH of 10). As used herein, the phrase “soluble” shall refer to greater than 60% solubility at room temperature in an aqueous solution

Embodiments also include methods for the preparation of isolates of such fractions. The term “isolates”, as used herein, such as in the case of a “protein isolate” shall refer to protein-containing compositions having >70 wt. % protein.

Protein isolate herein can have varying solubility. In various embodiments, the protein can have a solubility of at least about 400 g/L, 450 g/L, 500 g/L, 550 g/L, 600 g/L, 650 g/L, 700 g/L, 750 g/L, 800 g/L, 850 g/L, or 900 g/L in water at 25 degrees Celsius.

The color of protein isolate can be measured in various ways. In some embodiments, a Hunter Colorimeter can be used to measure color values L* (Whiteness or Brightness), The value for L* vary from 100 (White) to 0 (Black). The higher the L* value the brighter and whiter the color.

In some embodiments, the black-eyed pea protein isolates can have a brightness (L*) values of 65, 70, 75, 80, 85. In some embodiments, the brightness (L*) value can be in a range wherein any of the foregoing can serve as the upper or lower bound of the range. In an embodiment, the brightness (L*) values can be up to 70, 75, 80, and 85.

Protein digestibility-corrected amino acid score (PDCAAS) is a method of evaluating the protein quality based on both the amino acid requirements of humans and their ability to digest it. See, e.g., Schaafsma, J. Nutr., vol. 130 no. 7 1865S-1867S (Jul. 1, 2000). A value of 1 is the highest and a value of 0 is the lowest. The formula for calculating PDCAAS is as follows:

$$PDCAAS ( \% ) = \frac{mg of limiting amino acid in 1 g of test protein}{mg of same amino acid in 1 g of reference protein} \times fecal true digestibility ( \% ) \times 100$$

Protein isolates herein can have a relatively high PDCAAS value. In some embodiments, the protein isolate has a PDCAAS of at least about 0.55. In some embodiments, the protein isolate has a PDCAAS of at least about 0.60. In some embodiments, the protein isolate has a PDCAAS of at least about 0.70.

Protein isolates herein can have varying molecular weights. In some embodiments, the protein can have an average molecular weight of greater than or equal to 10,000, 15,000, 20,000, 25,000, 30,000, 35,000, 40,000, or 45,000 Daltons. In some embodiments, the protein can have an average molecular weight of less than or equal to 90,000, 85,000, 80,000, 75,000, 70,000, 65,000, 60,000, or 55,000 Daltons. In some embodiments, the protein can have an average molecular weight falling within a range wherein any of the foregoing weights can serve as the upper or lower bound of the range. In some embodiments, the protein can have an average molecular weight of about 30,000 to about 70,000 Daltons.

Protein isolates herein can have a clean taste profile. One technique for assessing this in the context of such protein isolates is to determine the odor threshold value for H₂S, methanethiol, dimethyl disulfide, dimethyl trisulfide, and hexanal. A Static Headspace Gas Chromatography/Mass Spectroscopy/Olfactometry (GC/MS/O) method (as described in Lei, Q. and W. L. Boatright, 2001. Compounds Contributing to the Odor of Aqueous Slurries of Soy Protein Concentrates, Journal of Food Science, 66(9):1306-1310; and Boatright, W. L., 2002. Effect of Gallic Acid on the Aroma Constituents of Soymilk and Soy Protein Isolates, Journal of the American Oil Chemists' Society, 79(4):317-323) can be used to measure these primary odorants in proteins. The formula for odor threshold values is as follows:

Odor Value in Air=Concentration in Headspace/Published Odor Threshold.

The odor threshold value>10 is considered significant. In embodiments herein, the black-eyed pea protein has an odor threshold value of less than 10 for one or more of H₂S, methanethiol, dimethyl disulfide, dimethyl trisulfide, and hexanal.

### Methods of Producing Protein Isolates

Included herein are methods of making black-eyed pea protein products including, but not limited to, methods of making black-eyed pea protein isolates.

Preparation

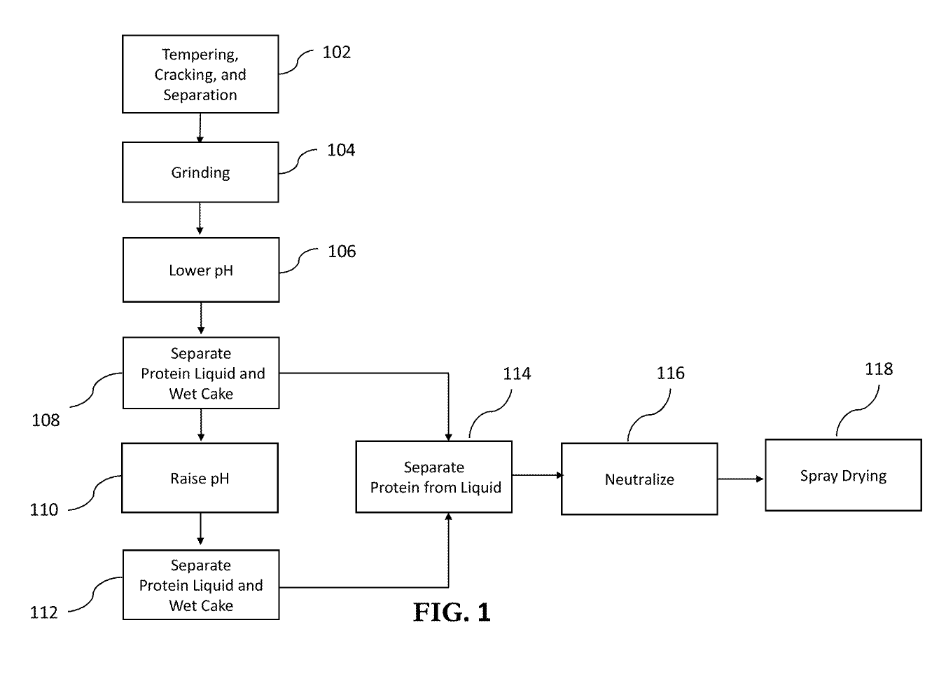

Various steps can be taken to initially process the black-eyed peas. In some embodiments, a step of tempering, cracking, and hull separation can be included. In various embodiments, a step of grinding dehulled black-eyed peas can be included. Referring to FIG. 1, the method can include an operation of tempering, cracking, and hull separation 102. Tempering can be performed by adding water to raise the moisture content to 35 to 60 by wt. percent moisture. In some embodiments, tempering whole black-eyed pea seeds can include adding water to raise the moisture content to 35 to 45 wt. percent moisture.

In some embodiments, cracking the tempered black-eyed pea seeds with a shearing mill (or mill exhibiting shearing action) to loosen meat-hull mixture. In some embodiments, the mill exhibiting shearing action can be selected from the group consisting of a cracking roller mill and a disc-mill.

In some embodiments, no more than 120, 90, 60, 45, 30 or 15 minutes elapses between exposing the whole black-eyed peas seeds to water and subsequent cracking of the black-eyed pea seeds. In some embodiments, the amount of time between exposing the whole black-eyed peas seeds to water and subsequent cracking of the black-eyed pea seeds falls within a range between any of the foregoing amounts of time.

The method can also include separating the meat from the hulls in the meat-hull mixture. Separating the meat from the hulls in the meat-hull mixture can be performed with various pieces of equipment. In some embodiments, separating the meat from the hulls can be performed by at least one piece of equipment selected from a gravity table and an aspirator.

The method can also include an operation of grinding 104. Grinding can be performed with various pieces of equipment including, but not limited to, a FitzMill, a Quadro Comil mill or the like. In some embodiments, grinding can specifically include wet grinding the separated meat to a fine mash.

Acid Extraction

The method can also include an operation of lowering the pH 106. In various embodiments, the method can include lowering the pH of a composition comprising black-eyed pea mash to a pH of less than about 4.0, 3.5, 3.0, 2.8, 2.6, 2.4, 2.2, 2.0, 1.8, 1.6, 1.4, 1.2 or 1.0, or to a pH falling within a range between any of the foregoing. In some embodiments the pH is lowered to about 2.0 in an aqueous solvent. In various embodiments, the method can include lowering the pH by adding a food grade acid. Food grade acids can include, but are not limited to, acetic acid, propionic acid, sorbic acid, benzoic acid, succinic acid, adipic acid, fumaric acid, lactic acid, malic acid, tartaric acid, citric acid, and phosphoric acid. In various embodiments, the method can include lowering the pH by adding a weak acid. Weak acids herein can include, but are not limited to, phosphoric acid. In some embodiments, the black-eyed pea mash is held at a relatively low pH for an extended time. For example, the black-eyed pea mash can be held at a pH of about 1.8 to 2.2 (or 2.0) in an aqueous solvent for at least about 30, 45, 60, 75, or 90 minutes.

The method can also include an operation of separating an acidic wet cake out from the acidic protein liquid portion 108. Separating an acidic wet cake out from the liquid portion can be performed with various equipment. By way of example, the acidic wet cake can be separated from the liquid portion using a 2-phase decanter. In some embodiments, the acidic wet cake can be separated from the liquid portion using a 3-phase centrifuge. In some embodiments, the acidic wet cake can be separated from the liquid portion using a 2-phase decanter followed by using a 3-phase centrifuge.

The % extraction of protein in acid portion can vary. However, in some embodiments, the extraction of protein is at least about 50-55% by weight as a percent of total black-eyed pea seed protein.

Alkaline Extraction

The method can further include sequential alkaline extraction to recover remaining protein. For example, an operation of raising the pH 110 can be included. In some embodiments, starting with the acidic wet cake, the pH of the acidic wet cake can be raised to a pH of about 9, 9.5, 9.8, 10, 10.2, 10.4, 10.6, 10.8, 11 or to a pH falling within a range between any of the foregoing. In some embodiments, the pH is raised to 9.8 to 10.2 in an aqueous solvent. Raising the pH can be performed by adding a base, such as a strong base. In some embodiments, raising the pH is performed by adding a strong base selected from the group consisting of NaOH and KOH. In some embodiments, the resuspended wet cake is held at a relatively high pH for an extended time. For example, the resuspended wet cake mash can be held at a pH of about 10.0 in an aqueous solvent for at least about one hour.

The method can further include an operation of separating a basic wet cake out from the basic liquid portion 112. Separating the basic wet cake out from the liquid portion can be performed with various equipment. By way of example, the basic wet cake can be separated from the liquid portion using a 2-phase decanter. In some embodiments, the basic wet cake can be separated from the liquid portion using a 3-phase centrifuge. In some embodiments, the basic wet cake can be separated from the liquid portion using a 2-phase decanter followed by using a 3-phase centrifuge.

The % extraction of protein in alkaline portion can vary. However, in some embodiments, the extraction of protein is at least about 35-40% by weight as a percent of total seed protein from black-eyed pea material.

Further Processing

The method can also include an operation of combining acid protein fraction and alkaline protein fraction, separating and concentrating the protein from the liquid 114. In some embodiments, separation of protein is achieved by isoelectric precipitation. In some embodiments, concentrating soluble protein is achieved by microfiltration.

In some embodiments, the total combined protein extraction can be from 85%-95% by weight as a percent of total seed protein from black-eyed pea material.

In some embodiments, the total protein can be further processed. For example, the protein can be neutralized using a strong base 116. In some embodiments, a concentrate can be formed containing at least about 70 wt. % protein. In some embodiments, the method can further include an operation of spray drying black-eyed protein isolate 118.

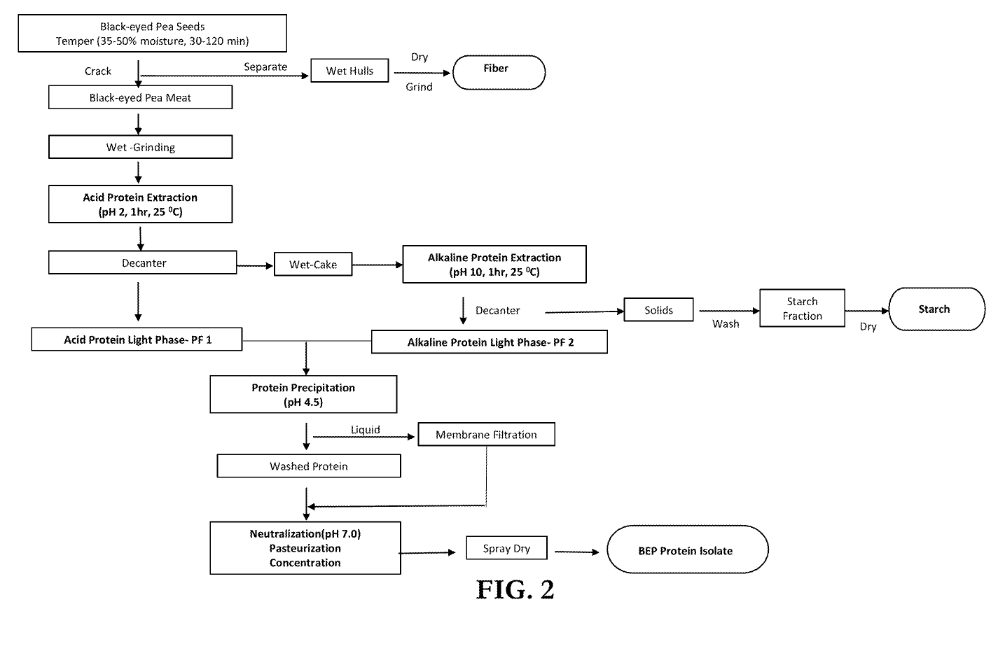

In an embodiment, a method of producing a black-eyed pea protein isolate is included herein. The method can include tempering black-eyed pea seeds to 35-45 wt. % moisture content and holding 30-120 min, cracking the tempered seeds to loosen the hull and the meat, and separating these two fractions. The method can further include wet-grinding the meat fraction to a finer mash. The method can further include lowering the pH of the black-eyed pea mash to about 2.0 in an aqueous solvent. The method can further include separating an acidic wet cake out from the acidic protein liquid fraction. The method can further include raising the pH of the acidic wet cake to about 10 in an aqueous solvent. The method can further include separating alkaline wet cake out from the alkaline protein liquid fraction. The method can further include combining acid and alkali protein liquid fractions to provide a total protein. The method can further include isoelectric precipitation, concentration, pasteurization, and spray drying. Aspects of steps performed in accordance with some embodiments herein are shown in FIG. 2.

### Food Products and Beverages

Black-eyed pea protein products herein may be used for protein fortification in high-acid juice based beverages, allergen free non-dairy, non-soy low acid beverages, plant-based yogurts, bakery products, baked snacks, cream soups, meat analogs, cheese analogs and frozen desserts. However, the applications for black-eyed pea protein products herein are not so limited. Products herein can include, but are not limited to, various food products and beverages. Beverages can include, but are not limited to, high-acid beverages, neutral beverages, carbonated beverages, non-carbonated beverages, high protein beverages, meal replacement beverages, nutraceutical beverages, and the like. Food products can include, but are not limited to soups, baked products, breads, crackers, cookies, bars, meal replacement products, snacks, baked snacks, and the like.

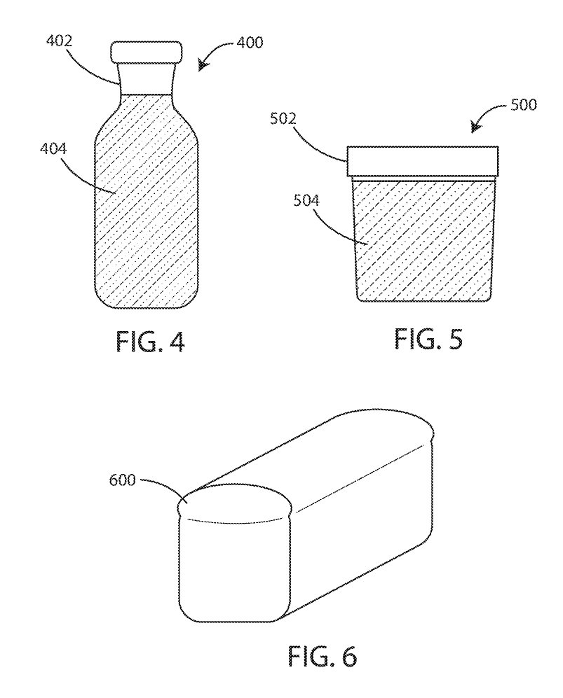

Referring now to FIG. 4, a schematic view is shown of a beverage 400 in accordance with various embodiments herein. The beverage 400 can include a container 402 and food material 404 disposed within the container 402. The food material 404 can be any of the beverages described above. In some embodiments, the beverage 400 can include at least about 0.1, 0.5, 1, 1.5 or 2 grams of protein per fluid ounce. In an embodiment, the beverage 400 can be at a pH of about 3.5 to about 4.0. In an embodiment, the beverage can be at a pH of about 6.5 to about 7.0. In some embodiments, at least 40, 50, 60 70, 80, 90, 95, or 99% by weight of the protein in the beverage 400 is from black-eyed pea protein. The black-eyed pea protein can have a PDCAAS of at least about 0.60.

Referring now to FIG. 5, a schematic view is shown of a food product 500 in accordance with various embodiments herein. The food product 500 can include a container 502 and a food material 504 disposed within the container 502. The food material 504 can be any of the food materials described above. The food product 500 can have at least about 1, 2, 3, 4, 5, 6, 7, 8, 9, or 10 grams of protein per serving. In some embodiments, at least about 50, 60, 70, 75, 80, 85, 90 or 95% by weight of the protein in the food product 500 is from black-eyed pea protein. The black-eyed pea protein can have a PDCAAS of at least about 0.60.

Referring now to FIG. 6, a schematic view is shown of a food product 600 in the form of a baked product in accordance with various embodiments herein. The food product 600 can have at least about 1, 2, 3, 4, 5, 6, 7, 8, 9, or 10 grams of protein per serving. In some embodiments, at least about 50, 60, 70, 75, 80, 85, 90 or 95% by weight of the protein in the food product 600 is from black-eyed pea protein. The black-eyed pea protein can have a PDCAAS of at least about 0.60.

It will be appreciated that food products and beverages herein can include various components other than the protein isolate including, but not limited to, water, juice, flour, fats including but not limited to vegetable oils, sweeteners including but not limited to sugar, leavening agents, salts, carbonation, plant extracts, anti-foaming agents, nutrient, natural and/or artificial flavoring agents, preservatives, coloring agents, and the like.

In addition, methods of making food products and beverages are included herein. Methods can include various steps including one or more of mixing ingredients, pasteurizing and/or sterilizing, baking, carbonating, leavening, packaging, bottling, and the like.

Aspects may be better understood with reference to the following examples. These examples are intended to be representative of specific embodiments, but are not intended as limiting the overall scope of embodiments herein.

### EXAMPLES

### Example 1—Effect of Tempering Moisture and Time

In this example, the effect of tempering moisture and time were demonstrated for optimal dehulling of black-eyed pea seeds. Black-eyed pea seeds (5 kg each) were tempered to varying moisture (30, 35, 40, 45, 50% by weight) content by adding predetermined amount of water. Seeds and water were mixed in a ribbon blender for 5 minutes. The seeds were rested for 30 min, 60 min, 120 min, and 12 hours. The tempered seeds were passed through a cracking roller (or a disc mill) at a pre-determined gap setting. The samples were collected for each of the moisture and time conditions. Samples were evaluated for cracking efficiency, degree of hull separation from meat, and fines. The results are shown below in the Table 1. The optimal cracking and hull separation were observed with 35-45% by weight moisture and shorter tempering times (30 min−120 min).

| Moisture | De-hulling Efficiency - Tempering Time | | | | |
|---|---|---|---|---|---|
| (wt. %) | 30 min | 60 min | 2 hr | 12 hr | Observations |
| 30 | Poor | Poor | Poor | Poor | No clean |
| | | | | | separation |
| | | | | | of hulls |
| | | | | | from meat. |
| | | | | | Intact hulls |
| | | | | | sticking to |
| | | | | | meat. |
| 35 | Good | Good | Good | Poor | Good hull |
| | | | | | separation |
| | | | | | with short |
| | | | | | tempering |
| | | | | | times. |
| 40 | Good | Good | Good | Poor | Good hull |
| | | | | | separation |
| | | | | | with short |
| | | | | | tempering |
| | | | | | times. |
| 45 | Good | Good | Good | Poor | Good hull |
| | | | | | separation |
| | | | | | with short |
| | | | | | tempering |
| | | | | | times. |
| 50 | Good | Good | Good | Poor | Good hull |
| | | | | | separation |
| | | | | | with short |
| | | | | | tempering |
| | | | | | times. |

### Example 2—Separation of Cracked Black-Eyed Pea Seeds into Hull and Meat Fractions

In this example, separation of cracked black-eyed pea seeds into hull and meat fractions using gravity table was demonstrated.

Black-eyed pea samples were prepared as follows: Seeds were soaked in excess water (1:5) for 30 minutes, de-hulled using disc mill and then both meat and hulls were dried to a target moisture content (MC by weight) (Control −58% MC, 50% MC, 40% MC and 30% MC). 25-35 kg samples were prepared for each condition. Gravity table parameters (length angle: 5.9°; width angle: 2.8°; speed of the table: 588 cycles per min; air velocity: 3400 fpm) were set to obtain clean separation of hulls from meat. End angle plate was adjusted 45°-80° to get a two-way separation. The results are shown below in the Table 2. Good separation results were observed with the seeds with moisture content of 30-40 wt. % moisture resulting in 93% by weight meat yield with hardly any hulls in the meat (<1% by weight).

| | Fraction Yields (Kg, %) | | | |
|---|---|---|---|---|
| Variables | Hulls | Meat | Total | Observations |
| 30% MC | 1.86 (7%) | 23.56 (93%) | 25.42 (100%) | Single pass, |
| | | | | Good separation, |
| 40% MC | 1.90 (7%) | 24.68 (93%) | 26.58 (100%) | Single pass, |
| | | | | Good separation, |
| 50% MC* | 1.30 (5%) | 24.00 (95%) | 25.30 (100%) | Double pass, not |
| | | | | good separation, |
| | | | | hulls tend to stick |
| 58% MC* | 3.04 (9%) | 31.06 (91%) | 34.10 (100%) | Double pass, not |
| | | | | good separation, |
| | | | | hulls tend to stick |

### Example 3—Extraction of Protein

This example illustrates the preparation of dried black-eyed pea protein products in accordance with various embodiments of the invention and FIG. 2.

200 kg of dry black-eyed pea seeds were tempered to 35 wt. % moisture by adding appropriate amount of water and mixing them together in a ribbon blender and held for 30 minutes. The tempered seeds were passed through a disc mill to split the seeds and loosen the hull. The meat-hull mixture was passed through an aspirator and separated into black-eyed pea meat and hulls.

The de-hulled black-eyed pea meat was wet-milled to a fine paste using Fitz mill and mixed with water (1:3) to form a slurry. The slurry was then adjusted to pH 2 (1.9-2.1) with phosphoric acid (85% aqueous solution) and continually stirred for 1 hour at room temperature (25° C.). The slurry was separated using a decanter into acid protein containing light phase fraction (APF) and acid-wet cake. The acid protein containing liquid fraction (PF-1) was set aside.

The acid-wet cake was suspended in water (1:2), adjusted to pH 10 (9.9-10.1) with NaOH (50% aqueous solution) and continually stirred for 1 hour at 25° C. The slurry was separated using a decanter to alkaline protein containing light phase (PF-2) and wet cake. The protein fractions PF-1 and PF-2 were combined, and pH was adjusted to 4.5 with phosphoric acid (85% aqueous solution) and stirred for 30 minutes to form protein precipitate. The precipitated protein was separated from the liquid, washed. and collected. The protein in liquid portion, that is not precipitated by iso-electric precipitation was further recovered using membrane filtration process. All proteins fractions were combined, adjusted to 10 wt. % solids, and pH to 7.0 with NaOH (50% aqueous solution), pasteurized, and spray dried into a protein isolate powder. This process resulted in extracting and recovering at least 85-90% by weight of total seed protein from the raw materials. Black-eyed pea protein isolate produced contained ≥70% protein, by weight.

### Example 4—Extraction of Protein

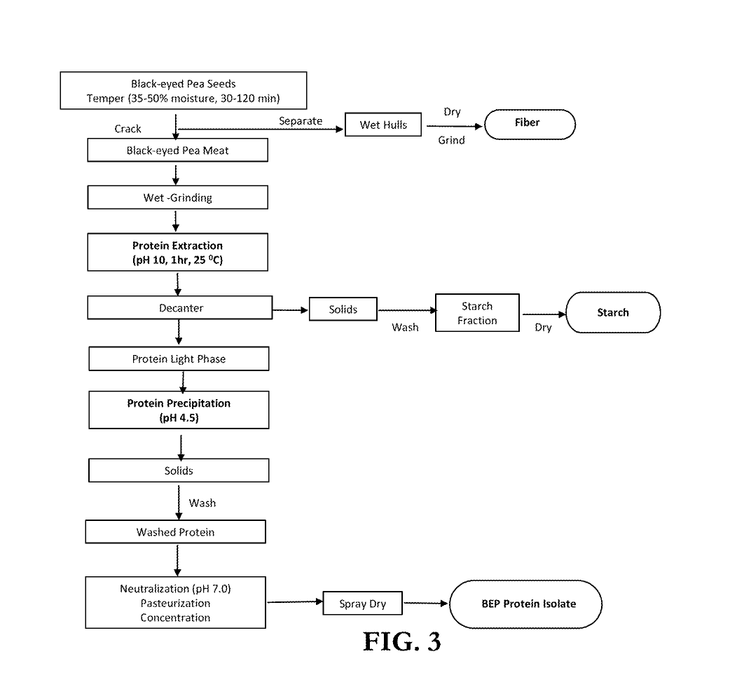

This example illustrates an alternate method for extracting and producing black-eyed pea protein isolate from black-eyed peas in accordance with various embodiments and FIG. 3.

In this experiment, de-hulled black-eyed pea meat (200 kg) was used as starting material for protein extraction. The black-eyed pea meat was wet-milled into a fine paste using a Fitz mill and mixed with water (1:3) to form a slurry. The pH of the slurry was adjusted to 10 with NaOH (50% aqueous solution) and continually stirred for 1 hour at 25° C. The slurry was separated using a decanter into a protein containing light phase and wet cake. The pH of the light phase was adjusted to pH 4.5 with phosphoric acid (85% aqueous solution) and stirred for 30 minutes to form protein precipitate. The precipitated protein was separated from the liquid, washed, and solids were adjusted to 10% and pH to 7.0 with NaOH (50% aqueous solution), pasteurized, and spray dried into a protein isolate powder. This process resulted in extracting and recovering only 60-65% of total protein from the seeds by weight. Black-eyed pea protein isolate contained 70% protein, by weight.

### Example 5—Functional Properties of Black-Eyed Pea Protein Vs. Other Proteins

In this example, the functional properties of black-eyed pea protein are characterized and compared against other legume proteins (soy and pea). The results are show in Table 3. Black-eyed pea protein has functional properties comparable to pea or soy protein.

| | Black-eyed | | |
|---|---|---|---|
| Functional Property | Pea Protein | Pea Protein | Soy Protein |
| Water Hydration | 4.0 | 3.34 | 12.4 |
| (g water/g protein) | | | |
| Oil Holding (g oil/g protein) | 1.66 | 1.12 | 1.85 |
| Emulsification Capacity | 147 | 133 | 175 |
| (g oil/g protein) | | | |
| Foaming Capacity (%) | 138 | 65 | 170 |
| Solubility (%) | | | |
| pH 2.0 | 63 | 23 | 22 |
| pH 4.0 | 11 | 18 | 4 |
| pH 6.0 | 10 | 18 | 15 |
| pH 8.0 | 78 | 18 | 24 |
| pH 10.0 | 91 | 38 | 33 |

### Example 6—Evaluation of Primary Odorants

This example illustrates the comparison of primary odorants present in black-eyed pea protein and several other plant proteins.

A Static Headspace Gas Chromatography/Mass Spectroscopy/Olfactometry (GC/MS/O) method (described above) was used to measure the primary odorants. An Agilent 6890 Gas Chromatograph/5973 Mass Spectrometer configured with 30 meter column with MS-No Vent & Cryogenic Trap (<−85° C.) for Olfactometry was used to perform static headspace analysis.

A 5 wt. % slurry of various proteins such as soy, pea, rice, and black-eyed pea was prepared and 15 ml of head space from protein slurry was injected into the GC column using a 25 mL gas tight syringe with an on/off valve that was preheated to 45° C. The column was a DB5-MS capillary column (30 m×0.53 mm i.d.) with 1.5 um film thickness. The column temperature was held at 35° C. for 4 minutes, then increased at 15° C./min to 250° C. and held for 5 minutes. The electron ionization detector was set to perform a scan of ions from 33-350 m/z. Concentration of the select headspace compounds (mg/m³) were estimated. The analytical results are presented in the table 4 below. The values in the parenthesis represents the Odor Value in Air, which is calculated as the ratio of Concentration in Headspace/Published Odor Threshold. Odor values>10 are considered significant and contributors to the odor. As results indicate, black-eyed pea protein found to contain very low concentration of odorants, when compared to soy, rice, and pea proteins. Results also show that soy, rice and pea has significant amounts of odor causing compounds such as hydrogen sulfide (H₂S), methanethiol (MT), dimethyl disulfide, dimethyl trisulfide, and hexanal.

| | PROTEIN SOURCE | | | | | | | |
|---|---|---|---|---|---|---|---|---|
| COM- | Black-eyed | | | | | | | |
| POUND | Pea | | Soy | | Rice | | Pea | |
| H₂S | n.d | | 0.426 | (61) | n.d | | n.d | |
| Acetal- | n.d | | n.d | | 0.271 | (9) | n.d | |
| dehyde | | | | | | | | |
| MT | n.d | | 0.131 | (82) | 0.014 | (9) | 0.009 | (5) |
| 2-Ethyl | n.d | | n.d | | 0.62 | | 0.832 | |
| Furan | | | | | | | | |
| Pentanal | 0.076 | (2) | n.d | | n.d | | 0.257 | (8) |
| DMDS | 0.015 | (0.5) | 0.014 | (0.5) | 0.341 | (12) | 0.030 | (1) |
| Hexanal | 0.312 | (7) | 0.127 | (3) | 0.886 | (15) | 2.264 | (53) |
| Benzal- | 0.0633 | (0.6) | n.d | | n.d | | 0.073 | (1) |
| dehyde | | | | | | | | |
| DMTS | n.d | | n.d | | 0.021 | (18) | n.d | |
| 2-Pentyl | 0.292 | (3) | 0.092 | (1) | 0.825 | (9) | 3.54 | (39) |
| Furan | | | | | | | | |

Note:
H₂S = hydrogen Sulfide;
MT = methanethiol;
DMDS = dimethyl disulfide;
DMTS = dimethyl trisulfide

### Example 7—Cream Soup Emulsions

This example illustrates the application of black-eyed pea protein in cream soup emulsions.

A cream of mushroom soup formulation was used to evaluate emulsion stability and overall acceptability of black-eyed pea protein. Two soups—a control soup with soy protein concentrate emulsion, and a test soup with black-eyed pea protein as emulsifier were prepared. The emulsions were prepared by blending protein, oil, and water in 1:10:30 weight ratio and homogenizing at 500/2500 psi. The emulsions were blended with mushrooms, thickeners and flavorings, weighed into cans, sealed, and retort processed at 250° F. for 60 minutes. The test soup made with black-eyed pea protein emulsion was found to be acceptable with clean taste and preferred over the control soup made with soy protein.

### Example 8—Fortified White Bread

This example illustrates the fortification of white bread with black-eyed pea protein.

A white bread formulation was used to fortify with black-eyed pea protein. Two bread doughs were prepared: (1) Control dough—no protein fortification (3 g protein per serving); (2) Test dough—with black-eyed pea protein (7 g of protein per serving). In the test dough, wheat flour was replaced with black-eyed pea protein isolate (70 wt. % protein) by 10 wt. %. The other optional ingredients in the formulation include: salt, sugar, yeast, oil, non-fat dry milk, and water. The doughs were prepared, weighed, formed, placed in pans, proofed, and baked at 420° F. for 25-30 min. Breads were evaluated by the team. Bread fortified with black-eyed pea protein had good loaf volume, texture, and clean taste and comparable to the control bread, while delivering the protein benefit.

### Example 9—Fortified Crackers

This example illustrates the fortification of crackers with black-eyed pea protein.

A cracker formulation was used to fortify with black-eyed pea protein. Two cracker doughs were prepared: (1) Control dough—no protein fortification (3 g protein per serving); (2) Test dough—with black-eyed pea protein (7 g protein per serving). In the test dough, wheat flour was replaced with black-eyed pea protein isolate (70 wt. % protein) by 35 wt. %. The other optional ingredients in the formulation include: salt, sugar, yeast, spice flavoring, oil, cheese, leavening agents (sodium bicarbonate, ammonium bicarbonate) and water. The doughs were mixed, fermented for 4-5 hours at room temperature, sheeted, cut into desired shapes, and baked. Baked crackers were seasoned with oil and topical salt. Crackers fortified with black-eyed pea protein have good texture, good puff (volume), and clean taste, while delivering the protein benefit.

### Example 10—Plant-Based Yogurt

This example illustrates the preparation of a plant-based yogurt with black-eyed pea protein.

A plant-based greek style yogurt formulation was prepared as follows: First, a black-eyed pea protein milk was prepared by hydrating black-eyed pea protein isolate (70 wt. % protein) at 9.25 wt. % in hot water (150-160° F.) for 10-15 minutes using a high shear mixer. To the hydrated the protein, canola oil (3 wt. %), sugar (5 wt. %) and further mixed for 5 minutes. The mixture was then homogenized at 500/2500 psi, pasteurized (195° F. for 1 minute), and cooled to 100° F. A vegan yogurt culture at 0.01 wt. % was added and incubated at 95° F. overnight (12 hours). After 12 hour fermentation, the set yogurt was stirred and filled into containers. The yogurt delivers 10 g protein per 150 g serving and had a nice creamy texture and clean taste.

### Example 11—Plant-Based Milk Beverage

This example illustrates the preparation of a plant-based milk beverage with black-eyed pea protein.

A plant-based milk formulation was prepared as follows: First, a black-eyed pea protein milk was prepared by hydrating black-eyed pea protein isolate (70 wt. % protein) at 5.5% by weight (10 g protein per serving) in hot water (150-160° F.) for 10-15 minutes using a high shear mixer. To the hydrated the protein, canola oil (1.4 wt. %), sugar (3 wt. %), flavorings, and further mixed for 5 minutes. The mixture was then homogenized at 500/2500 psi to a uniform emulsion. It is then aseptically processed (285° F. for 30 sec) using Microthermics processor.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a task or adopt a configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Aspects have been described with reference to various specific and preferred embodiments and techniques. However, many variations and modifications may be made while remaining within the spirit and scope herein.

1. A method of producing a black-eyed pea protein isolate comprising:
tempering whole black-eyed pea seeds by adding water to raise the moisture content to 35 to 60 by wt. percent moisture;
cracking the tempered black-eyed pea seeds with a shearing mill to loosen meat-hull mixture, wherein no more than 120 minutes elapses between exposing the whole black-eyed peas seeds to water and subsequent cracking of the black-eyed pea seeds;
separating the meat from the hulls in the meat-hull mixture;
wet grinding the separated meat to a fine mash;
forming a slurry with water and lowering the pH to 1 to 3 to solubilize protein;
separating acid protein-rich liquid portion from the protein-starch containing mixture;
suspending acid wet cake into aqueous suspension and raising the pH to 9 to 11 to solubilize protein:
separating alkaline-protein rich liquid fraction out from protein-starch containing mixture; and
combining acid protein-rich fraction and alkaline protein-rich fraction and precipitating the protein out of the liquid portion to form a protein isolate.

2. (canceled)

3. The method of claim 1, wherein no more than 120 minutes elapses between exposing the whole black-eyed peas seeds to water and subsequent cracking of the black-eyed pea seeds.

4-5. (canceled)

6. The method of claim 1, wherein cracking is performed by the mill exhibiting shearing action selected from the group consisting of a cracking roller mill and a disc-mill.

7. (canceled)

8. The method of claim 1, wherein adjusting the pH to 1 to 3 is performed by adding a weak acid.

9. (canceled)

10. The method of claim 1, wherein adjusting the pH to 1 to 3 comprises adjusting the pH to 1.8 to 2.2.

11. The method of claim 1, wherein adjusting the pH to 9 to 11 is performed by adding a strong base.

12. (canceled)

13. The method of claim 1, wherein adjusting the pH to 9 to 11 comprises adjusting the pH to 9.8 to 10.2.

14-17. (canceled)

18. The method of claim 1, wherein at least 90 wt. % of the meat in the seeds is recovered by separating the meat from the hulls in the meat-hull mixture.

19. (canceled)

20. The method of claim 1, wherein the meat contains <1 wt. % of hulls and black specs.

21. The method of claim 1, wherein the protein isolate contains at least 70 wt. % protein.

22-23. (canceled)

24. The method of claim 1, wherein at least 90 wt. % of total seed protein is recovered.

25. The method of claim 1, wherein the protein isolate has brightness (L*) value of at least 75.

26. The method of claim 1, wherein the protein isolate has an average molecular weight of about 30,000 to about 70,000 Daltons.

27. The method of claim 1, wherein the protein isolate has a solubility of at least about 600 g/L in water at 25 degrees Celsius.

28. The method of claim 1, wherein the protein isolate has odor threshold value of <10 for hexanal.

29. The method of claim 1, wherein the protein isolate has odor threshold value of <10 for at least one sulfur containing odorant selected from the group consisting of H₂S, methanethiol, dimethyl disulfide, and dimethyl trisulfide.

30. (canceled)

31. A black-eyed pea protein isolate comprising:
at least about 70% by weight protein;
having a brightness (L*) value of at least 75.

32. The protein isolate of claim 31, wherein the protein isolate has odor threshold value of <10 for hexanal.

33. The protein isolate of claim 31, wherein the protein isolate has odor threshold value of <10 for at least one sulfur containing odorant selected from the group consisting of H₂S, methanethiol, dimethyl disulfide, and dimethyl trisulfide.

34. The protein isolate of claim 31, wherein the black-eyed pea protein has an average molecular weight of about 30,000 to about 70,000 Daltons.

35-40. (canceled)
